(19) United States
(12) Patent Application Publication
(10) Pub. No.: US 2026/0335203 A1
(43) Pub. Date: Oct. 29, 2026

(54) METHOD FOR PRODUCING OILY DISPERSION AND COSMETIC FORMULATED WITH OILY DISPERSION

(71) Applicant: NIKKO CHEMICALS CO., LTD., Tokyo (JP)

(72) Inventors: Noboru NAGATANI, Tokyo (JP); Shohei NAKAMURA, Tokyo (JP); Junya NAKANO, Tokyo (JP); Meng QIN, Tokyo (JP)

(21) Appl. No.: 19/123,112

(22) PCT Filed: Sep. 4, 2023
(86) PCT No.: PCT/JP2023/032178

§ 371 Date: Apr. 22, 2025

(30) Foreign Application Priority Data
Nov. 2, 2022 (JP) 2022-176426

(51) Int. Cl. A61K 8/29; A61K 8/22; A61K 8/37

(52) U.S. Cl. CPC A61K 8/29; A61K 8/22; A61K 8/37; A61K 2800/412

(57) ABSTRACT

An inorganic oxide powder which is easily aggregated has a feature of high dispersibility in an oil. The oily dispersion provides a cosmetic having good dispersion stability. A method for producing an oily dispersion containing an inorganic oxide powder and an ester oil of a fatty acid with a carbon number of 8 to 22, the method comprising: a step (1) of adding the inorganic oxide powder to the ester oil while heating the ester oil at a temperature of 70 °C to 120 °C; and a step (2) of further mixing a dispersion to which the total amount of the inorganic oxide powder is added at a temperature of 70 °C to 120 °C.

### TECHNICAL FIELD

The present invention relates to a method for producing an oily dispersion and a cosmetic formulated with the oily dispersion. More specifically, the present invention relates to a method for producing an oily dispersion capable of being dispersed in an oil even at a high solid content by adding an inorganic oxide powder to an ester oil of a fatty acid at a specific temperature, and a cosmetic obtained by formulating the oily dispersion produced by the production method.

### BACKGROUND ART

In general, in an oily dispersion for cosmetics, pigments to be formulated in makeup cosmetics such as foundation, eyeshadow, and blusher, or basic cosmetics such as sunscreen cosmetics, hair cosmetics, milky lotions, and creams are highly dispersed in an oily medium in advance. For this reason, oily dispersions for cosmetics are widely used because of good handling properties such as less scattering and contamination of pigments during preparation of cosmetics, and no special mixing equipment because the pigments are already highly dispersed.

In addition, since the inorganic oxide powder has a strong aggregation force between the powders, it is difficult to disperse the inorganic oxide powder in the oil agent as it is. Therefore, the pigment used in the oily dispersion is widely subjected to a hydrophobic treatment for the purpose of facilitating dispersion of the inorganic oxide powder in the oil, and for example, a method is known in which the surface of the pigment is coated with a higher fatty acid, a higher alcohol, a hydrocarbon, or a silicone such as a silicone oil, or a silicone resin to impart hydrophobicity. Among them, many methods for hydrophobic treatment of a pigment using silicones as surface treatment agents have been established so far (See, for example, Patent Literatures 1 and 2), and oily dispersions using them are also widely known (See, for example, Patent Literatures 3, 4, and 5).

Furthermore, since the inorganic oxide powder has a strong aggregation force between powders, in order to suppress re-aggregation after dispersion and maintain dispersion stability, it is common to add a silicone-based dispersant such as polyether-modified silicone, a surfactant, or the like to disperse the inorganic oxide powder (See, for example, Patent Literatures 6 and 7).

### CITATION LIST

### Patent Literatures

Patent Literature 1: JP 60-163973 A
Patent Literature 2: JP 62-177070 A
Patent Literature 3: JP 2002-80748 A
Patent Literature 4: JP 2005-171145 A
Patent Literature 5: JP 2011/007668 A
Patent Literature 6: JP 2007-291379 A
Patent Literature 7: JP 2012-184178 A

### SUMMARY OF INVENTION

However, in the method of hydrophobizing the inorganic oxide powder, an oily dispersion cannot be prepared unless the inorganic oxide powder is once subjected to a step of surface-treating the inorganic oxide powder with a surface treatment agent, and there is a problem in complexity.

In addition, when an oily dispersion obtained using a dispersant, a surfactant, or the like is formulated in a cosmetic, there is a possibility that an adverse effect such as deterioration of storage stability and makeup durability of the cosmetic is caused.

The present invention has been made in view of the above-described problems, and an object of the present invention is to provide a method for producing an oily dispersion for obtaining an oily dispersion having excellent dispersibility of an inorganic oxide powder without requiring a surface treatment in advance for enhancing dispersibility of the inorganic oxide powder in an oil and without adding a dispersant, and a cosmetic containing the oily dispersion produced by the production method.

### Solution to Problem

As a result of intensive studies to achieve the above object, the present inventors have found that an oily dispersion having high dispersibility of powder can be obtained by adding an inorganic oxide powder while heating an ester oil of a fatty acid with a carbon number of 8 to 22 at a temperature of 70 °C to 120 °C, and an oily dispersion capable of maintaining high dispersibility of pigment can be obtained even without using a dispersant, thereby completing the present invention.

A method for producing an oily dispersion according to an embodiment of the present invention includes: a step (1) of gradually adding an inorganic oxide powder to an ester oil while heating the ester oil of a fatty acid with a carbon number of 8 to 22 at a temperature of 70 °C to 120 °C; and a step (2) of further mixing a dispersion to which the total amount of the inorganic oxide powder is added at a temperature of 70 °C to 120 °C.

In the method for producing an oily dispersion of the present invention, it is preferable to use a kneading mixer or a wet mixing disperser for dispersion in the step (2) of the production method.

One aspect of the method for producing an oily dispersion of the present invention is a method for producing an oily dispersion, including: cooling the oily dispersion and further dispersing the oily dispersion using a wet mixing disperser after all the steps of the production method including the step (1) of adding an inorganic oxide powder while heating an ester oil of a fatty acid with a carbon number of 8 to 22 at a temperature of 70 °C to 120 °C and the step (2) of further mixing a dispersion to which the total amount of the inorganic oxide powder is added at a temperature of 70 °C to 120 °C.

The method for producing an oily dispersion of the present invention is preferably produced by cooling the dispersion to room temperature (20 °C to 30 °C) and further dispersing the dispersion using a wet mixing disperser after the step (2) of the above production method.

In the above aspect, the solid content concentration of the inorganic oxide powder at the time of producing the oily dispersion is preferably 25 mass % or more.

Furthermore, the cosmetic of the present invention is a cosmetic in which an oily dispersion produced by the production method of the present invention is formulated.

### DESCRIPTION OF EMBODIMENTS

Next, specific embodiments of a method for producing an oily dispersion according to the present invention and a cosmetic containing an oily dispersion produced by this production method will be described. In the present specification, “X to Y” indicating a range means “X or more and Y or less”. Unless otherwise specified, the operation is performed under the conditions of room temperature (20 °C to 30 °C)/relative humidity 45 to 55% RH. Further, all embodiment combinations should be understood to be disclosed in this application.

The method for producing an oily dispersion of the present invention includes a step (1) of adding an inorganic oxide powder to an ester oil while heating the ester oil of a fatty acid with a carbon number of 8 to 22 at a temperature of 70 °C to 120 °C, and a step (2) of further mixing a dispersion obtained by adding the total amount of the inorganic oxide powder at a temperature of 70 °C to 120 °C.

According to the present invention, by gradually adding an inorganic oxide powder to an ester oil of a fatty acid with a carbon number of 8 to 22 while heating at a temperature of 70 °C to 120 °C, an oily dispersion having high dispersibility of pigment and an oily dispersion capable of maintaining high dispersibility of powder without further using a dispersant are obtained, and a cosmetic containing the oily dispersion can be obtained.

The ester oil of a fatty acid with a carbon number of 8 to 22 (Hereinafter, it is also simply referred to as ester oil) used as a dispersion medium is an ester oil in which a carbon number of a portion derived from the fatty acid is 8 to 22. When a plurality of fatty acid-derived moieties such as diesters and triesters are present, it means that the carbon number of at least one fatty acid is 8 to 22, and preferably the carbon number of all fatty acids is 8 to 22. For example, tri (caprylic/capric)glyceryl is an ester of caprylic acid (C8)/capric acid (C10) and glycerin, and thus has a carbon number of 8 and a carbon number of 10 in the fatty acid moiety. The ester oil of a fatty acid with a carbon number of 8 to 22 is preferably a non-volatile, liquid or paste-like ester oil having fluidity at 25 °C. Here, the term “non-volatile” means that it does not volatilize at 25 °C, and is, for example, a component having a boiling point exceeding 250 °C at normal pressure. An ester oil of a fatty acid with a carbon number of less than 8 is not preferable because an oily dispersion having good dispersibility of an inorganic oxide powder cannot be obtained. In addition, an oily dispersion having good dispersibility of the inorganic oxide powder cannot be obtained even with a hydrocarbon oil or a silicone oil other than the ester oil. The ester oil of a fatty acid with a carbon number of 8 to 22 is more preferably an ester of a fatty acid with a carbon number of 8 to 18. The fatty acid may be a saturated fatty acid or an unsaturated fatty acid, but is preferably a saturated fatty acid, and the saturated fatty acid may be linear or branched.

Examples of such an ester oil include monoester oils such as isononyl isononanoate, isodecyl isononanoate, isotridecyl isononanoate, tricyclodecane methyl isononanoate, coco-caprylate/caprate, hexyl laurate, methylheptyl laurate, caprylyl laurate, decyl laurate, isopropyl myristate, methylheptyl myristate, 2-hexyldecyl myristate, octyldodecyl myristate, isopropyl palmitate, methylheptyl palmitate, 2-ethylhexyl palmitate, 2-ethylhexyl stearate, ethyl isostearate, isopropyl isostearate, isobutyl isostearate, decyl isostearate, methylheptyl isostearate, 2-hexyldecyl isostearate, 2-ethylhexyl hydroxystearate, octyldodecyl oleate, oleyl oleate, octyldodecyl ricinoleate, octyl p-methoxycinnamate, alkyl benzoate, and glyceryl isostearate; diester oils such as di(caprylic/capric) propanediol, neopentyl glycol diisononanoate, neopentyl glycol dicaprate, glyceryl diisostearate, polyglyceryl diisostearate, propanediol diisostearate, di(phytosteryl/octyldodecyl) lauroyl glutamate, di(cholesteryl/behenyl/octyldodecyl) lauroyl glutamate, di(octyldodecyl/phytosteryl/behenyl) lauroyl glutamate, and neopentyl glycol dioctanoate; triester oils such as trimethylolpropane triisostearate, diglyceryl triisostearate, diglyceryl triisostearate, caprylic/capric triglyceride, glyceryl trioctanoate, macadamia nut oil, olive oil, castor oil, jojoba oil, avocado oil, and sunflower oil; and pentaerythrityl tetraoctanoate, diglyceryl tetraisostearate, dipentaerythrityl tetraisostearate, and pentaerythrityl tetraisostearate.

Among them, the ester oil is preferably at least one selected from the group consisting of coco-caprylate/caprate, isopropyl myristate, 2-ethylhexyl palmitate, 2-ethylhexyl stearate, decyl isostearate, methyl heptyl isostearate, diglyceryl triisostearate, glyceryl tri (caprylic acid/capric acid), jojoba oil, diglyceryl tetraisostearate, and dipentaerythrityl tetraisostearate from the viewpoint of the low viscosity, good usability and the effect of the present invention. From the viewpoint of storage stability, the ester oil of fatty acid preferably contains tri (caprylic/capric)glyceryl.

In the present invention, examples of the inorganic oxide powder include inorganic oxides such as anhydrous silicic acid (silicon oxide), aluminum oxide, iron oxide (Red iron oxide, yellow iron oxide, black iron oxide, and the like), titanium oxide, zinc oxide, cerium oxide, and bismuth oxide which are generally used in cosmetics, and in particular, for the purpose of the ultraviolet ray shielding effect, examples thereof include inorganic oxides such as fine particles of titanium oxide, zinc oxide, cerium oxide, and bismuth oxide, and composites thereof. Among them, the inorganic oxide powder is preferably titanium oxide and/or iron oxide. As a preferred aspect, the inorganic oxide is an inorganic oxide powder which is not subjected to a fatty acid treatment. In another preferred aspect, the inorganic oxide powder is not subjected to hydrophobic surface treatment. Here, the hydrophobic treatment refers to treatment with a known hydrophobic surface treatment agent such as silicone treatment or fatty acid treatment. By using the inorganic oxide that has not been subjected to the hydrophobic treatment as described above, the dispersibility is maintained over time. The inorganic oxide powder is preferably an untreated powder. The untreated powder refers to a powder that is not surface-treated with a surface treatment agent.

The particle size of the inorganic oxide is, for example, 0.001 to 50 μm and may be 0.01 to 30 μm. The particle size is based on volume, and can be measured using a laser diffraction particle size distribution meter.

The solid content concentration of the inorganic oxide powder of the oily dispersion is preferably 25 mass % or more, more preferably 25 to 80 mass %, still more preferably 30 to 80 mass %, still more preferably 40 to 80 mass %, and particularly preferably 40 to 70 mass %. When the solid content concentration of the inorganic oxide powder is 25 mass % or more, dispersibility of the inorganic oxide powder after time has passed is improved, and when the solid content concentration is 80 mass % or less, aggregation of the powder is easily suppressed.

The method for producing an oily dispersion of the present invention is a production method carried out in the order of the following step (1) and step (2).

### Step (1);

In the step (1), while an ester oil of a fatty acid with a carbon number of 8 to 22 is heated at a temperature of 70 °C to 120 °C, an inorganic oxide powder is added to the ester oil, and all target charging amounts are added. In addition, it is preferable to gradually add an inorganic oxide powder to the ester oil.

The temperature of the ester oil is preferably 70 °C to 100 °C from the viewpoint of the effect of the present invention. The temperature of the ester oil is preferably maintained within the above temperature range while the inorganic oxide powder is added. It is preferable to appropriately perform heating using a heating means so that the temperature is maintained within the above temperature range.

The time for adding the inorganic oxide powder to the ester oil is, for example, 1 minute to 100 minutes.

Examples of the dispersion method for obtaining an oil dispersion in the step (1) include a method in which an inorganic oxide powder is charged while an ester oil is stirred using a stirrer such as a homomixer or a dispersion mixer (disper). The rotation speed at the time of stirring is not particularly limited as long as dispersion proceeds well, and is, for example, 1000 rpm to 5000 rpm.

From the viewpoint of the effect of the present invention, the mixing mass ratio of the ester oil to the inorganic oxide powder is preferably ester oil: inorganic oxide powder=75:25 to 20:80, more preferably 70:30 to 20:80, still more preferably 60:40 to 20:80, and particularly preferably 60:40 to 30:70.

From the viewpoint of the effect of the present invention, it is preferable not to use a dispersant in step (1). Here, examples of the dispersant include those listed below.

### Step (2);

The oil dispersion obtained in the step (1) is further mixed at a temperature of 70 °C to 120 °C to obtain an oily dispersion. The dispersion time in the step (2) is preferably 2 hours or more from the viewpoint of dispersibility of the inorganic oxide powder in the ester oil, and more preferably 2 hours to 10 hours, and still more preferably 2 to 5 hours from the viewpoint of saturation of the effect.

In the dispersion step, the dispersion may be continuously performed using a homomixer, a disper, or the like. However, it is preferable to use a kneading mixer capable of applying a higher shear force, for example, a kneading mixer such as kneader kneading, Henschel kneading, roll kneading, or extruder kneading, or a wet mixing disperser such as a propeller mixer, a high speed mixer, a dissolver, an Altimizer, a wet jet mill, a colloid mill, a masscolloider, a (wet) bead mill, a sand mill, a basket mill, or an AD mill.

In the step (2), a dispersant may be used. Examples of the dispersant include polyhydroxystearic acid, glyceryl hydroxystearate, polyglyceryl diisostearate-2, polyglyceryl triisostearate-2, polyglyceryl tetraisostearate-2, erythrityl triethylhexanoate, and sorbitan sesquiisostearate, and one or two or more of these can be used. Among these dispersants, polyhydroxystearic acid is most preferably used. From the viewpoint of dispersibility, the amount of the dispersant used is, for example, 1 part by mass to 10 parts by mass with respect to 100 parts by mass of the inorganic oxide.

In the steps (1) and (2), it is preferable to heat the ester oil and the oil dispersion so that the temperature of the ester oil and the oil dispersion is 70 °C to 120 °C as the temperature condition at the time of production. When the temperature is lower than 70 °C, the inorganic oxide powder cannot be uniformly dispersed in the ester oil, which is not preferable. When the temperature exceeds 120 °C, the ester oil is decomposed, and yellow iron oxide and black iron oxide in the inorganic oxide powder are discolored, which is not preferable.

The mixing temperature of the oil dispersion is preferably 70 °C to 100 °C from the viewpoint of the effect of the present invention. It is preferable that the temperature is in the above temperature range during mixing, and it is preferable that the temperature range is maintained while being appropriately heated using a heating means.

It is preferable that after the steps (1) and (2), the oily dispersion is cooled and a dispersion step is further added, because the inorganic oxide powder is further microparticulated and more uniformly dispersed in the oil. Cooling is preferably performed until the temperature reaches 40 °C or lower, and cooling is preferably performed until the temperature reaches room temperature (20 °C to 30 °C). Examples of cooling is performed by a method in which the stirring state is continued and the mixture is allowed to stand; a method in which the oily dispersion is cooled using a cooling means while the stirring state is continued and the like.

The disperser used in the dispersion after cooling is preferably a wet mixing disperser from the viewpoint of improving the dispersibility of the inorganic oxide powder in the oily dispersion, and above all, a wet bead mill is preferable. Specific examples of the wet mixing disperser are as described above. The dispersion time in the dispersion after cooling is, for example, 15 minutes to 5 hours, and may be 30 minutes to 3 hours.

When the oily dispersion is formulated into a cosmetic in the present invention, a dispersant can be used in a step for producing the oily dispersion within a range not affecting the storage stability and makeup durability of the cosmetic. Examples of the dispersant include polyhydroxystearic acid, glyceryl hydroxystearate, polyglyceryl diisostearate-2, polyglyceryl triisostearate-2, polyglyceryl tetraisostearate-2, erythrityl triethylhexanoate, and sorbitan sesquiisostearate, and one or two or more of these can be used. Among these dispersants, polyhydroxystearic acid is most preferably used.

Next, a cosmetic according to the present invention will be described. Since the cosmetic of the present invention uses an ester oil having high safety by formulating the oily dispersion prepared by the above-described production method, the cosmetic has good compatibility with other cosmetic components, has good stability after time has passed with a high solid content concentration, and has a good feel, and can produce a cosmetic such as a sunscreen having a high SPF value and high transparency. Specific examples of the cosmetic include makeup cosmetics such as foundation, eyeshadow, and blusher, and basic cosmetics such as sunscreen cosmetics, hair cosmetics, milky lotions, creams and the like. The formulating amount of the oily dispersion is not particularly limited, but is preferably 0.1 to 90 mass % in the cosmetic.

Furthermore, in the cosmetic of the present invention, components usually used in cosmetics, for example, powders, surfactants, oils, gelling agents, polymers, beauty ingredients, moisturizers, pigments, preservatives, fragrances, and the like can be used as long as the effects of the present invention are not impaired.

As the form of the cosmetic of the present invention, any dosage form such as a powder form, a milky lotion form, a cream form, a stick form, a solid form, a spray, a multi-layer separation type and the like may be used.

### EXAMPLE

Next, the present invention will be described more specifically based on Examples and Comparative Examples of the oily dispersion produced by the production method of the present invention and the cosmetic containing the oily dispersion, but the present invention is not limited to the following Examples.

### Example 1

2-Ethylhexyl palmitate, 40 g, was heated to 90 °C. After confirming that the temperature had reached 90 °C, 60 g of titanium oxide (particle size: 0.2 μm) was added little by little over 10 minutes while stirring 2-ethylhexyl palmitate with a disper at 2000 rpm. After the total amount of titanium oxide was charged, the mixture was further stirred at a temperature of 90 °C and with a disper at 2000 rpm for 3 hours to obtain an oily dispersion of titanium oxide having a solid content concentration of 60 mass %.

### Example 2

An oily dispersion of red iron oxide having a solid content concentration of 60 mass % was obtained in the same manner as in Example 1 except that red iron oxide (particle size: 0.5 μm) was used instead of titanium oxide.

### Example 3

An oily dispersion of yellow iron oxide having a solid content concentration of 60 mass % was obtained in the same manner as in Example 1 except that yellow iron oxide (particle size: 0.7 μm) was used instead of titanium oxide.

### Example 4

An oily dispersion of black iron oxide having a solid content concentration of 60 mass % was obtained in the same manner as in Example 1 except that black iron oxide (particle size: 0.2 μm) was used instead of titanium oxide.

### Example 5

The oily dispersion obtained in Example 1 was allowed to stand and cooled to room temperature (25 °C) while stirring was maintained, and further dispersed and mixed for 1 hour under the conditions of a peripheral speed of 10 m/s, q 0.5 mm of zirconia beads, and a filling rate of 70% using a wet bead mill (DYNO-MILL manufactured by SHINMARU ENTERPRISES CORPORATION) to obtain an oily dispersion of titanium oxide having a solid content concentration of 60 mass %.

### Example 6

Tri (caprylic/capric)glyceryl, 47 g, are heated to 90 °C. After confirming that the temperature has reached 90 °C, 50 g of fine particle titanium oxide (MT-05, manufactured by TAYCA CORPORATION) is added little by little over 9 minutes while stirring tri (caprylic/capric)glyceryl with a disper at 2000 rpm. The total amount of the fine particle titanium oxide was charged, 3 g of polyhydroxystearic acid (SALACOS HS-6C, manufactured by The Nisshin OilliO Group, Ltd.) was further added as a dispersant, and then the mixture was dispersed for 2 hours under the conditions of a peripheral speed of 10 m/s, a q 0.5 mm zirconia bead, a filling rate of 70%, and a temperature of 90 °C using a wet bead mill (DYNO-MILL manufactured by SHINMARU ENTERPRISES CORPORATION) to obtain an oily dispersion having a solid content concentration of fine particle titanium oxide of 50 mass %.

### Example 7

An oily dispersion of titanium oxide having a solid content concentration of 60 mass % was obtained in the same manner as in Example 1 except for using coco-caprylate/caprate instead of 2-ethylhexyl palmitate.

### Example 8

An oily dispersion of titanium oxide having a solid content concentration of 60 mass % was obtained in the same manner as in Example 1 except for using isopropyl myristate instead of 2-ethylhexyl palmitate.

### Example 9

2-Ethylhexyl stearate, 60 g, was heated to 80 °C. After confirming that the temperature had reached 80 °C, 40 g of fine particle zinc oxide (MZ-500, manufactured by TAYCA CORPORATION) was added little by little over 8 minutes while stirring 2-ethylhexyl stearate with a disper at 2000 rpm. After the total amount of zinc oxide was charged, the mixture was further stirred at a temperature of 80 °C and with a disper at 2000 rpm for 3 hours to obtain an oily dispersion of fine particle zinc oxide having a solid content concentration of 40 mass %.

### Example 10

Decyl isostearate, 50 g, was heated to 70 °C. After it was confirmed that the temperature reached 70 °C, 50 g of titanium oxide was added little by little over 9 minutes while stirring decyl isostearate with a disper at 2000 rpm. After the total amount of titanium oxide was charged, the mixture was further stirred at a temperature of 70 °C and with a disper at 2000 rpm for 3 hours to obtain an oily dispersion of titanium oxide having a solid content concentration of 50 mass %.

### Example 11

Methyl heptyl isostearate, 30 g, was heated to 100 °C. After it was confirmed that the temperature reached 100 °C, 70 g of titanium oxide was added little by little over 11 minutes while stirring methyl heptyl isostearate with a disper at 2000 rpm. After the total amount of titanium oxide was charged, the mixture was further stirred at a temperature of 100 °C and with a disper at 2000 rpm for 3 hours to obtain an oily dispersion of titanium oxide having a solid content concentration of 70 mass %.

### Example 12

An oily dispersion of titanium oxide having a solid content concentration of 50 mass % was obtained in the same manner as in Example 10 except that diglyceryl triisostearate was used instead of decyl isostearate.

### Example 13

An oily dispersion of titanium oxide having a solid content concentration of 70 mass % was obtained in the same manner as in Example 11 except for using jojoba oil instead of methyl heptyl isostearate.

### Example 14

An oily dispersion of titanium oxide having a solid content concentration of 60 mass % was obtained in the same manner as in Example 1 except that diglyceryl tetraisostearate was used instead of 2-ethylhexyl palmitate.

### Example 15

An oily dispersion of titanium oxide having a solid content concentration of 60 mass % was obtained in the same manner as in Example 1 except for using dipentaerythrityl tetraisostearate instead of 2-ethylhexyl palmitate.

### Example 16

2-Ethylhexyl palmitate, 40 g, was heated to 90 °C. After confirming that the temperature had reached 90 °C, 60 g of titanium oxide (particle size: 0.2 μm) was added little by little over 10 minutes while stirring 2-ethylhexyl palmitate with a disper at 2000 rpm. After the total amount of titanium oxide was charged, the mixture was dispersed for 2 hours under the conditions of a peripheral speed of 10 m/s, a @ 0.5 mm zirconia bead, a filling rate of 70%, and a temperature of 90 °C using a wet bead mill (DYNO-MILL manufactured by SHINMARU ENTERPRISES CORPORATION) to obtain an oily dispersion of titanium oxide having a solid content concentration of 60 mass %.

### Example 17

2-Ethylhexyl palmitate, 70 g, was heated to 90 °C. After confirming that the temperature reached 90 °C, 30 g of titanium oxide was added little by little over 5 minutes while stirring 2-ethylhexyl palmitate with a disper at 2000 rpm. After the total amount of titanium oxide was charged, the mixture was further stirred at a temperature of 90 °C for 3 hours to obtain an oily dispersion of titanium oxide having a solid content concentration of 30 mass %.

### Comparative Example 1

2-Ethylhexyl palmitate, 40 g, was heated to 60 °C. After confirming that the temperature reached 60 °C, 60 g of titanium oxide was added little by little over 10 minutes while stirring 2-ethylhexyl palmitate with a disper at 2000 rpm, and the total amount of titanium oxide was charged to obtain an oily dispersion of titanium oxide having a solid content concentration of 60 mass %.

### Comparative Example 2

2-Ethylhexyl palmitate, 40 g, was heated to 90 °C. After confirming that the temperature had reached 90 °C, 60 g of hydrophobized titanium oxide surface-treated with stearic acid (2% by mass with respect to titanium oxide) was added little by little over 10 minutes while stirring 2-ethylhexyl palmitate with a disper at 2000 rpm. After the total amount of the hydrophobized titanium oxide was charged, the mixture was further stirred at a temperature of 90 °C for 3 hours to obtain an oily dispersion of titanium oxide having a solid content concentration of 60 mass %.

### Comparative Example 3

An oily dispersion of titanium oxide having a solid content concentration of 60 mass % was obtained in the same manner as in Example 1 except for using hexyl hexanoate instead of 2-ethylhexyl palmitate.

### Comparative Example 4

An oily dispersion of titanium oxide having a solid content concentration of 60 mass % was obtained in the same manner as in Example 1 except for using squalane instead of 2-ethylhexyl palmitate.

### Comparative Example 5

An oily dispersion of titanium oxide having a solid content concentration of 60 mass % was obtained in the same manner as in Example 1 except for using dimethylpolysiloxane (6cs) instead of 2-ethylhexyl palmitate.

Next, a method for evaluating dispersion stability will be described.

Redispersibility (Shake well when particles are settled) of the sample after elapse of one day from the preparation of the sample and the sample stored at 40 °C for 1 month was visually observed.

Evaluation Criteria after One Day
◯: The dispersed particles were uniform and no aggregation was observed.
Δ: The dispersed particles were almost uniform, but slight aggregation was observed.
x: The dispersed particles were not uniform, and significant aggregation was observed.
   Evaluation Criteria after 1-Month Storage
◯: The dispersed particles were uniform and no aggregation was observed.
Δ: The dispersed particles were almost uniform, but slight aggregation was observed.
x: The particles were not redispersed, and settling or significant aggregation was observed.

The results of evaluating Examples 1 to 15 and Comparative Examples 1 to 6 by the above evaluation method are shown in Table 1.

TABLE 1

| | Example 1 | Example 2 | Example 3 | Example 4 | Example 5 | Example 6 |
|---|---|---|---|---|---|---|
| Dispersibility (after 1 day) | ◯ | ◯ | ◯ | ◯ | ◯ | ◯ |
| Dispersibility (40 °C, after 1 | Δ | Δ | Δ | Δ | ◯ | ◯ |
| month storage) | | | | | | |

| | Example 7 | Example 8 | Example 9 | Example 10 | Example 11 | Example 12 |
|---|---|---|---|---|---|---|
| Dispersibility (after 1 day) | ◯ | ◯ | ◯ | ◯ | ◯ | ◯ |
| Dispersibility (40 °C, after 1 | Δ | Δ | Δ | Δ | Δ | Δ |
| month storage) | | | | | | |

| | | | | | | Comparative |
|---|---|---|---|---|---|---|
| | Example 13 | Example 14 | Example 15 | Example 16 | Example 17 | Example 1 |
| Dispersibility (after 1 day) | ◯ | ◯ | ◯ | ◯ | ◯ | X |
| Dispersibility (40 °C, after 1 | Δ | Δ | Δ | ◯ | Δ | X |
| month storage) | | | | | | |

| | Comparative | Comparative | Comparative | Comparative |
|---|---|---|---|---|
| | Example 2 | Example 3 | Example 4 | Example 5 |
| Dispersibility (after 1 day) | ◯ | X | X | X |
| Dispersibility (40 °C, after 1 | X | X | X | X |
| month storage) | | | | |

Examples 1 to 15 of the present invention product had good dispersibility 1 day after production and 1 month after storage at 40 °C. In addition, it was found that the present invention product was excellent in dispersibility, particularly dispersibility after time has passed over time, as compared with Comparative Examples 1 to 6. In addition, it was also found that the oily dispersion of the present invention was also excellent in dispersibility over time as compared with the case of using an inorganic oxide powder surface-treated with a fatty acid in advance.

### Example 18: Oil-Based Foundation

The formulation described in Table 2 was dispersed and mixed with a homomixer to obtain an oil-based foundation. The dispersion stability of Example 18 was good (neither aggregation nor settling of powder occurred over time).

TABLE 2

| | Composition |
|---|---|
| Component | (mass %) |
| (1) Polyether modified silicone | 2.0 |
| (2) Decamethylcyclopentasiloxane | 57.65 |
| (3) Dimethylpolysiloxane (6cs) | 7.0 |
| (4) Alkyl acrylate/dimethicone copolymer | 0.5 |
| (5) Mica | 5.0 |
| (6) Tocopherol | 0.05 |
| (7) Ethylhexyl methoxycinnamate | 5.0 |
| (8) Nylon powder | 5.0 |
| (9) Titanium oxide oily dispersion of Example 5 | 15.0 |
| (10) Red iron oxide oily dispersion of Example 2 | 0.7 |
| (11) Yellow iron oxide oily dispersion of Example 3 | 2.0 |
| (12) Black iron oxide oily dispersion of Example 4 | 0.1 |

### Example 19: Sunscreen Cream

A sunscreen cream was obtained using the formulation composition described in Table 3 by the following production method. The dispersion stability of Example 19 was good.

TABLE 3

| | Composition |
|---|---|
| Component | (mass %) |
| (1) Polyglyceryl diisostearate | 3.0 |
| (2) Polyglyceryl polyricinoleate | 2.0 |
| (3) Oily dispersion of titanium dioxide particles | 36.0 |
| from Example 6 | |
| (4) Nylon powder | 5.0 |
| (5) Purified water | 45.5 |
| (6) 1,3-Butylene glycol | 6.0 |
| (7) Sodium chloride | 2.0 |
| (8) Preservative | 0.5 |

### (Production Method)

The components (1) to (4) are mixed. To the mixture, aqueous solutions of the components (5) to (8) which had been mixed and dissolved in advance were added while being stirred with a homomixer to obtain a target water-in-oil sunscreen cream.

In Example 18 and Example 19, the dispersion stability of the pigment was excellent in the cosmetics (no aggregation or settling of the powder occurred over time).

### INDUSTRIAL APPLICABILITY

The oily dispersion produced by the production method of the present invention has a feature that the inorganic oxide powder which is easily aggregated has high dispersibility in an oil. In addition, this oily dispersion can provide a cosmetic having good dispersion stability, and has high industrial applicability.

The present invention includes the following aspects.
(1) A method for producing an oily dispersion containing an inorganic oxide powder and an ester oil of a fatty acid with a carbon number of 8 to 22, the method including:
a step (1) of adding an inorganic oxide powder to the ester oil while heating the ester oil at a temperature of 70 °C to 120 °C; and
a step (2) of further mixing a dispersion to which the total amount of the inorganic oxide powder is added at a temperature of 70 °C to 120 °C.
(2) The method for producing an oily dispersion according to above (1), wherein a solid content concentration of the inorganic oxide powder in the oily dispersion is 25 mass % or more.
(3) The method for producing an oily dispersion according to above (1) or (2), wherein a kneading mixer or a wet mixing disperser is used for mixing in the step (2).
(4) The method for producing an oily dispersion according to any one of above (1) to (3), wherein the oily dispersion is cooled and further dispersed using a wet mixing disperser after the step (2).
(5) The method for producing an oily dispersion, wherein the oily dispersion is cooled and further dispersed using a wet mixing disperser after all steps of the production method according to above (3).
(6) The method for producing an oily dispersion according to any one of above (1) to (5), wherein the oily dispersion is an oily dispersion containing only two components of an inorganic oxide powder and an ester oil of a fatty acid with a carbon number of 8 to 22.
(7) The method for producing an oily dispersion according to any one of above (1) to (6), wherein the inorganic oxide powder is not hydrophobized.
(8) A cosmetic formulating an oily dispersion produced by the production method according to any one of above (1) to (7).

The present application is based on Japanese Patent Application No. 2022-176426 filed on Nov. 2, 2022, the disclosure content of which is incorporated herein by reference in its entirety.

1. A method for producing an oily dispersion containing an inorganic oxide powder and an ester oil of a fatty acid with a carbon number of 8 to 22, the method comprising:
a step (1) of adding the inorganic oxide powder to the ester oil while heating the ester oil at a temperature of 70 °C to 120 °C; and
a step (2) of further mixing a dispersion to which a total amount of the inorganic oxide powder is added at a temperature of 70 °C to 120 °C.

2. The method for producing an oily dispersion according to claim 1, wherein a solid content concentration of the inorganic oxide powder in the oily dispersion is 25 mass % or more.

3. The method for producing an oily dispersion according to claim 1, wherein a kneading mixer or a wet mixing disperser is used for mixing in the step (2).

4. The method for producing an oily dispersion according to claim 1, wherein the oily dispersion is cooled and further dispersed using a wet mixing disperser after the step (2).

5. The method for producing an oily dispersion according to claim 3, wherein an oily dispersion is cooled and further dispersed using a wet mixing disperser after all steps of the production method according to claim 3.

6. The method for producing an oily dispersion according to claim 1, wherein the oily dispersion is an oily dispersion containing only two components of an inorganic oxide powder and an ester oil of a fatty acid with a carbon number of 8 to 22.

7. The method for producing an oily dispersion according to claim 3, wherein the oily dispersion is an oily dispersion containing only two components of an inorganic oxide powder and an ester oil of a fatty acid with a carbon number of 8 to 22.

8. The method for producing an oily dispersion according to claim 4, wherein the oily dispersion is an oily dispersion containing only two components of an inorganic oxide powder and an ester oil of a fatty acid with a carbon number of 8 to 22.

9. The method for producing an oily dispersion according to claim 5, wherein the oily dispersion is an oily dispersion containing only two components of an inorganic oxide powder and an ester oil of a fatty acid with a carbon number of 8 to 22.

10. The method for producing an oily dispersion according to claim 1, wherein the inorganic oxide powder is not hydrophobized.

11. A cosmetic comprising an oily dispersion produced by the production method according to claim 1.

12. A cosmetic comprising an oily dispersion produced by the production method according to claim 3.

13. A cosmetic comprising an oily dispersion produced by the production method according to claim 4.

14. A cosmetic comprising an oily dispersion produced by the production method according to claim 5.
